(19) United States
(12) Patent Application Publication
(10) Pub. No.: US 2009/0269409 A1
(43) Pub. Date: Oct. 29, 2009

(54) PHARMACEUTICAL COMPOSITIONS COMPRISING ESZOPICLONE

(76) Inventors: Mukesh Kumar Garg, Hyderabad (IN); Parag Prakash Borde, Jalna (IN); Pradeep Kumar Gupta, Indore (IN)

Correspondence Address: DR. REDDY''S LABORATORIES, INC., 200 SOMERSET CORPORATE BLVD, SEVENTH FLOOR, BRIDGEWATER, NJ, 08807-2862, US

(21) Appl. No.: 12/428,818

(22) Filed: Apr. 23, 2009

Related U.S. Application Data
(60) Provisional application No. 61/081,829, filed on Jul. 18, 2008.

(30) Foreign Application Priority Data
Apr. 24, 2008 (IN) 1018/CHE/2008

(51) Int. Cl. A61K 9/14; A61K 31/4985

(52) U.S. Cl. 424/489; 514/249

(57) ABSTRACT

Pharmaceutical compositions comprising eszopiclone, including its pharmaceutically acceptable salts, hydrates, clathrates, solvates, polymorphs, and mixtures thereof. The invention also relates to processes for preparing the compositions and their methods of use.

The present invention relates to pharmaceutical compositions comprising eszopiclone, including its pharmaceutically acceptable salts, hydrates, clathrates, solvates, polymorphs, and mixtures thereof. The invention also relates to processes for preparing the compositions and their methods of use.

The present invention also relates stable pharmaceutical compositions of eszopiclone, and the processes for the preparation thereof.

Eszopiclone is a nonbenzodiazepine hypnotic agent that is a pyrrolopyrazine derivative of the cyclopyrrolone. It has a chemical name (+)-(5S)-6-(5-chloropyridin-2-yl)-7-oxo-6,7-dihydro-[3,4-b]pyrazin-5-yl-4-methylpiperazine-1-carboxylate. Its molecular weight is 388.81, and its empirical formula is C₁₇H₁₇ClN₆O₃. It has a single chiral center with an (S)-configuration, and the chemical structure of Formula I.

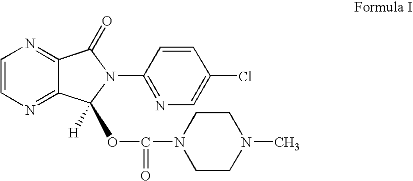

Eszopiclone is a white to light yellow crystalline solid. It is very slightly soluble in water; slightly soluble in ethanol, and soluble in phosphate buffer of pH 3.2. It is commercially available in LUNESTA™ tablets with the drug strengths 1, 2, and 3 mg, from Sepracor Inc.; the tablets contain the inactive ingredients calcium phosphate, colloidal silicon dioxide, croscarmellose sodium, hypromellose, lactose, magnesium stearate, microcrystalline cellulose, polyethylene glycol, titanium dioxide, triacetin, and FD&C Blue No. 2.

Eszopiclone is disclosed in U.S. Pat. Nos. 6,864,257, 6,444,673, 6,319,926, 6,436,936, and 6,638,535.

U.S. Patent Application Publication Nos. 2007/0054914, 2007/0098788, 2004/0147521, 2005/0222156, and 2004/0022844, and International Application Publication No. WO 2005/097132, disclose various compositions of eszopiclone.

LUNESTA is prescribed for the treatment of insomnia. LUNESTA, when administered at bedtime, decreases sleep latency and improves sleep maintenance.

Eszopiclone, being a hygroscopic molecule, is sensitive to various conditions such as thermal stress, oxidative stress, base hydrolysis and acid hydrolysis, photo-degradation, and water hydrolysis leading to formation of impurities.

Regulatory authorities worldwide require that the amounts of these impurities to be maintained at the lowest possible levels in pharmaceutical compositions and formulations. Hence there exists a need for stabilized compositions of eszopiclone or its salts.

### SUMMARY

The present invention relates to pharmaceutical compositions comprising eszopiclone or its pharmaceutically acceptable salts, hydrates, clathrates, solvates, polymorphs or mixtures thereof. The invention also relates to processes for preparing the compositions and methods of use thereof.

The present invention also relates to stable pharmaceutical compositions of eszopiclone, and to processes for the preparation thereof.

In an embodiment, the present invention includes stable pharmaceutical compositions comprising eszopiclone or its salts, wherein the compositions are stabilized by maintaining equilibrium relative humidity (ERH) of the compositions not higher than about 15%.

In an embodiment the present invention includes pharmaceutical compositions comprising eszopiclone or its salts, wherein the compositions comprise less than about 2%, or less than about 1%, or less than about 0.5%, of a 5-chloro-2-aminopyridine impurity, expressed by weight of the label escopiclone content.

In embodiments the present invention includes particle size distributions of eszopiclone or its salts, wherein D₉₀ is not more than about 20 μm, D₅₀ is not more than about 15 μm or not more than about 10 μm, and D₁₀ is not more than about 10 μm or not more than about 5 μm.

An embodiment of the present invention includes eszopiclone or its salts having bulk densities in the range of about 0.3 g/mL to about 0.8 g/mL, and tapped densities in the range of about 0.5 g/mL to about 0.9 g/mL.

In embodiments the present invention includes particle size distributions of final blends comprising eszopiclone and excipients, wherein about 0.01% to about 1% of particles have sizes greater than about 425 μm, about 1% to about 10% of particles have sizes greater than about 250 μm, about 20% to about 75% of particles have sizes greater than about 180 μm, and about 30% to about 100% of particles have sizes greater than about 150 μm.

In an embodiment the present invention includes bulk densities of final blends of eszopiclone and excipients for compression in the range of about 0.4 g/ml to about 0.8 g/ml, and tapped densities in the range of about 0.5 g/ml to about 0.9 g/ml.

### DETAILED DESCRIPTION

The present invention relates to pharmaceutical compositions comprising eszopiclone or its pharmaceutically acceptable salts, hydrates, clathrates, solvates, polymorphs or mixtures thereof. The invention also relates to processes for preparing the compositions and their methods of use.

The present invention also relates to stable pharmaceutical compositions of eszopiclone and the processes for the preparation thereof.

The number of people suffering from insomnia has increased in recent years due to stressful lifestyles.

The precise mechanism of eszopiclone action is not known, but its effect is believed to result from its interaction with GABA-receptor complexes at binding domains located close to, or allosterically coupled to, benzodiazepine receptors. Eszopiclone is a non-benzodiazepine hypnotic that is a pyrrolopyrazine derivative of the cyclopyrrolone class with a chemical structure unrelated to pyrazolo pyrimidines, imidazopyridines, benzodiazepines or other drugs with known hypnotic properties.

The term “pharmaceutically acceptable salt” refers to salts prepared from pharmaceutically acceptable non-toxic acids or bases, including inorganic acids and bases and organic acids and bases. Suitable pharmaceutically acceptable acids for forming salts with eszopiclone, without limitation thereto, include acetic, benzenesulfonic (besylate), benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, and p-toluenesulfonic acids.

Suitable bases that can used in the reaction include but are not limited to: alkali metal hydroxides such as lithium hydroxide, sodium hydroxide, potassium hydroxide and the like; carbonates of alkali metals such as sodium carbonate, potassium carbonate and the like; bicarbonates of alkali metals such as sodium bicarbonate, potassium bicarbonate, and the like; ammonia; and mixtures thereof. These bases can be used in the form of solids or in the form of aqueous solutions.

During the manufacturing process and during storage of a dosage form, eszopiclone is prone to a number of reactions such as thermal stress, oxidative stress, base hydrolysis, acid hydrolysis, photo-degradation, and water hydrolysis, any of which can give rise to impurities. Some of the impurities, which may be generated as a result of the manufacturing process and/or form during storage include:

1) The impurity having a chemical name 5-chloro-2-aminopyridine, which is formed due to presence of heat and moisture content in the pharmaceutical composition, is represented by Formula II.

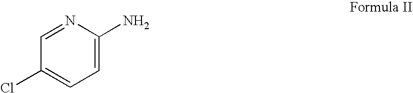

2) Impurity “A,” having a chemical name (5RS)-6-(5-chloropyridin-2-yl)-7-oxo-6,7-dihydro-5H-pyrrolo[3,4-b]pyrazin-5-yl 4-methylpiperazine-1-carboxylate-4-oxide, also known as “N-oxide zopiclone,” is formed by metabolism, is inactive, and is represented by Formula III.

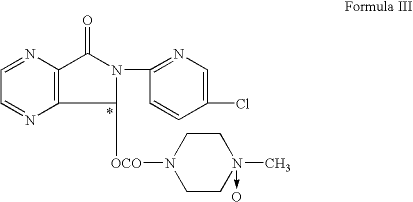

3) Impurity “B,” having a chemical name (7RS)-6-(5-Chloropyridin-2-yl)-7-hydroxy-6,7-dihydro-5H-pyrrolo[3,4-b]pyrazin-5-one, is formed during the penultimate synthesis, may preexist in starting material but can also be formed by hydrolysis of eszopiclone during storage, and is represented by Formula IV.

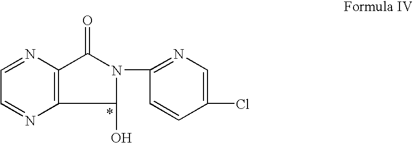

4) Impurity “C,” having a chemical name 6-(5-Chloropyridin-2-yl)-6,7-dihydro-5H-pyrrolo[3,4-b]pyrazin-5-one, is also known as the “deshydroxy impurity,” and is represented by Formula V.

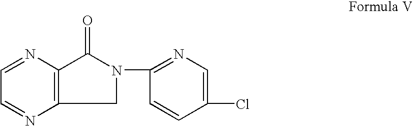

5) Impurity “D,” having a chemical name (−)-5R-6-(chloropyridin-2-yl)-7-oxo-6,7-dihydro-5H-pyrrolo[3,4-b]pyrazin-5-yl-4-methyl-piperazine-1 -carboxylate, is represented by Formula VI.

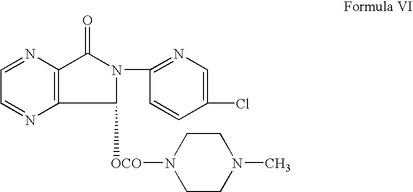

6) The “desmethyl impurity,” having a chemical name (5S)-6-(5-chloropyridin-2yl)-7-oxo-6,7-dihydro-5H-pyrrolo [3,4-b]pyrazin-5-yl-piperazine-1-carboxylate, is formed during the process of production of eszopiclone and is also known to be a degradation product, and is represented by Formula VII.

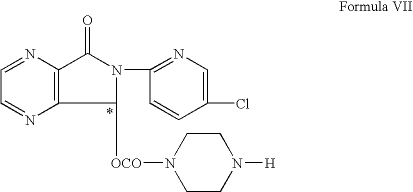

It has been observed that eszopiclone is highly susceptible to oxidative degradation (including by atmospheric oxygen) and acid hydrolysis with significant increase in the 5-chloro-2-aminopyridine content, thus 5-chloro-2-aminopyridine is a major impurity.

In an embodiment of the present invention, a residual solvent content in the pharmaceutical compositions is less than the limits set in ICH guidelines.

In an embodiment the present invention includes stable pharmaceutical compositions.

In another embodiment the present invention includes methods of stabilizing pharmaceutical compositions comprising eszopiclone, which methods include maintaining equilibrium relative humidity conditions for low in built moisture during processes of preparing the compositions.

Relative humidity is defined as the ratio of the partial pressure of water vapor in a gaseous mixture of air and water to the saturated vapor pressure of water at a given temperature. Relative humidity (RH) is expressed as a percentage and is calculated in the following manner:

$$R H = \frac{{p}_{( {H}_{2} O )}}{{p}_{( {H}_{2} O )}^{*}} \times 100 \%$$

where RH is the relative humidity of the mixture being considered; p(H₂O) is the partial pressure of water vapor in the mixture; and p*(H₂O) is the saturation vapor pressure of water at the temperature of the mixture.

Equilibrium Relative Humidity or “ERH” of a material is the relative humidity when the movement of moisture from a material to the environment (and vice versa) have equalized. This ERH balance is achieved when vapor pressures within the material and in the environment have equalized. At this point the moisture level of a material can be expressed in terms of ERH.

In embodiments the present invention includes pharmaceutical compositions containing eszopiclone wherein the ERH for said pharmaceutical compositions is less than about 15%, or less than about 10%, relative humidity.

In embodiments the present invention includes pharmaceutical compositions comprising eszopiclone or its salts, wherein said compositions comprise less than about 2%, or less than about 1%, or less than about 0.5%, of 5-chloro-2-aminopyridine, expressed as a percentage of the label eszopiclone content.

In embodiments the present invention includes pharmaceutical compositions comprising eszopiclone or its salts, wherein said compositions comprise less than about 2%, or less than about 1%, or less than about 0.5%, of impurity A, expressed as a percentage of the label eszopiclone content.

In embodiments the present invention includes pharmaceutical compositions comprising eszopiclone or its salts, wherein said compositions comprise less than about 2%, or less than about 1%, or less than about 0.5%, of impurity B, expressed as a percentage of the label eszopiclone content.

In embodiments the present invention includes pharmaceutical compositions comprising eszopiclone or its salts, wherein said compositions comprise less than about 2%, or less than about 1%, or less than about 0.5%, of impurity C, expressed as a percentage of the label eszopiclone content.

In embodiments the present invention includes pharmaceutical compositions comprising eszopiclone or its salts, wherein said compositions comprise less than about 2%, or less than about 1%, or less than about 0.5%, of impurity D, expressed as a percentage of the label eszopiclone content.

In embodiments the present invention includes pharmaceutical compositions comprising eszopiclone or its salts, wherein said compositions comprise less than about 2%, or less than about 1%, or less than about 0.5%, of the desmethyl impurity, expressed as a percentage of the label eszopiclone content.

Being a rather insoluble compound, it might be expected that particle size reduction would improve the solubiltiy of eszopiclone, as the decrease in particle size increases the surface area and thus increases the solublity. The present compositions involve a reduced particle size of eszopiclone to improve the dissolution profile.

A uniform distribution of the drug in the formulation may be achieved in many ways such as by using drug with uniform particle size distribution, or by optimizing different steps of processing of the composition such as mixing and blending the active and inactive excipients, or by selection of excipients, etc.

In embodiments the present invention provides pharmaceutical compositions comprising eszopiclone or its salts, wherein said compositions have content uniformity (uniformity of content) of eszopiclone such that the relative standard deviation is not more than about 5.

Various parameters impacting the compression process include the physical parameters of active agent as well as that of final blend. Physical parameters such as compressibility, flow, moisture content (determined, e.g., by Karl Fischer (KF) apparatus or infrared moisture balances), particle size (determined by sieve analyzers or a laser diffraction particle size analyzer), bulk density and tapped density, compressibility index, Hausner ratio (determined by USP density apparatus), flow properties (determined by Flowdex apparatus) and content of uniformity, etc.

In embodiments, the present invention provides pharmaceutical compositions comprising eszopiclone or its pharmaceutically acceptable salts, wherein said compositions have moisture contents less than about 8% w/w.

The particle size distribution of a material is generally described in terms of D₁₀, D₅₀, D₉₀, and D_{[4, 3]} used routinely to describe the particle sizes or size distribution. It is expressed as volume or weight or surface percentage. Dₓ as used herein is defined as the size of particles where x volume or weight percent of the particles have sizes less than the value given. D_{[4, 3]} is the volume mean diameter of the eszopiclone or a final blend with excipients for compression. D₉₀ for example means that 90% of the particles are below a stated particle size. D₅₀ is considered to be a mean or average particle size of a powder. Particle sizes or particle size distribution of the eszopiclone or final blend for compression of the present invention are determined by techniques that are known to the person skilled in the art, including but not limited to sieve analysis, size analysis by laser light diffraction such as using a Malvern particle size analyzer (Malvern Instruments Ltd., Malvern, Worcestershire, United Kingdom), and the like.

Generally, the eszopiclone that is used to prepare a composition will have an average particle size less than about 50 μm.

Embodiments of the present invention utilizee particle size distributions of the eszopiclone or its salts wherein D₉₀ is not more than about 10 μm, D₅₀ is not more than about 8 μm, and D₁₀ is not more than about 4 μm.

In embodiments the present invention includes particle size distributions of final blends comprising eszopiclone and excipients, wherein about 0.01% to about 1% of particles have sizes greater than about 425 μm, about 1% to about 10% of particles have sizes greater than about 250 μm, about 20% to about 75% of particles have sizes greater than about 180 μm, and about 30% to about 100% of particles have sizes greater than about 150 μm.

Other physicochemical characteristics of compositions are the density properties such as bulk and tapped density. Bulk density of a substance is the undisturbed packing density of that substance and tapped bulk density relates to the packing density after tapping a bed of substance until no change in the packing density is seen.

Bulk density and tapped density can be determined using compendial bulk density apparatus, such as the method given in United States Pharmacopeia 29, United States Pharmacopeial Convention, Inc., Rockville, Md., 2005, at pages 2638-2639).

Embodiments of the present invention provide bulk densities of eszopiclone or its salts in the range of about 0.4 g/mL to about 0.8 g/mL, and tapped densities in the range of about 0.6 g/mL to about 0.9 g/mL.

In embodiments the present invention includes bulk densities of final blends comprising eszopiclone and excipients for compression in the range of about 0.4 g/mL to about 0.8 g/mL, and tapped densities in the range of about 0.5 g/mL to about 0.9 g/mL.

In embodiments the present invention provides pharmaceutical compositions comprising eszopiclone or its salts, wherein the compositions are in solid oral dosage forms, such as tablets, capsules, lozenges, or pills.

The solid dosage forms may include pharmaceutical excipients, including but not limited to any one or more of fillers, binding agents, disintegrants, lubricating agents, taste-masking agents, preservatives, buffers, wetting agents, film forming agents and coloring agents.

### Fillers:

Various useful fillers or diluents include but are not limited to starches, lactose, mannitol (Pearlitol™ SD200), cellulose derivatives, confectioners sugar and the like. Different grades of lactose include but are not limited to lactose monohydrate, lactose DT (direct tableting), lactose anhydrous, Flowlac™ (available from Meggle Products), Pharmatose™ (available from DMV) and others. Different grades of starches include but are not limited to maize starch, potato starch, rice starch, wheat starch, pregelatinized starch (commercially available as PCS PC10 from Signet Chemical Corporation) and Starch 1500, Starch 1500 LM grade (low moisture content grade) from Colorcon, fully pregelatinized starch (commercially available as National 78-1551 from Essex Grain Products) and others. Different cellulose compounds that can be used include crystalline celluloss and powdered celluloses. Examples of crystalline cellulose products include but are not limited to CEOLUS™ KG801, Avicel™ PH101, PH102, PH301, PH302 and PH-F20, PH-1 12 microcrystalline cellulose 114, microcrystalline cellulose 112, Cellactose™ 80 (a spray-dried mixture of 75% alpha-lactose monohydrate and 25% cellulose powder, supplied by Molkerei MEGGLE Wasserberg GmbH & Co. KG, Wasserburg, Germany), and MicroceLac™ 100 (a spray-dried mixture of 75% alpha-lactose monohydrate and 25% microcrystalline cellulose, supplied by Molkerei MEGGLE Wasserberg GmbH & Co. KG, Wasserburg, Germany). Other useful diluents include but are not limited to carmellose, sugar alcohols such as mannitol (Pearlitol™ SD200), sorbitol and xylitol, calcium carbonate, magnesium carbonate, dibasic calcium phosphate, and tribasic calcium phosphate.

### Binders:

Various useful binders include but are not limited to hydroxypropylcelluloses (Klucel™ LF), hydroxypropylcelluloses (Klucel EXF) hydroxypropyl methylcelluloses or hypromelloses (Methocel™), polyvinylpyrrolidones or povidones (PVP-K25, PVP-K29, PVP-K30, PVP-K90), Plasdone™ S 630 (copovidone), powdered acacia, gelatin, guar gum, carbomers (e.g. Carbopol™), methylcelluloses, polymethacrylates, and starches.

### Disintegrants:

Various useful disintegrants include but are not limited to carmellose calcium (Gotoku Yakuhin Co., Ltd.), carboxymethylstarch sodium (Matsutani Kagaku Co., Ltd., Kimura Sangyo Co., Ltd., etc.), croscarmellose sodium (Ac-di-sol™ from FMC-Asahi Chemical Industry Co., Ltd.), crospovidones, examples of commercially available crospovidone products including but not limited to crosslinked povidone, Kollidon™ CL (manufactured by BASF, Germany), Polyplasdone™ XL, XI-10, and INF-10 (manufactured by ISP Inc., USA), and low-substituted hydroxypropyl celluloses. Examples of low-substituted hydroxypropylcellulose include but are not limited to low-substituted hydroxypropylcellulose LH11, LH21, LH31, LH22, LH32, LH20, LH30, LH32 and LH33 (all manufactured by Shin-Etsu Chemical Co., Ltd.). Other useful disintegrants include sodium starch glycolate, colloidal silicon dioxide (e.g., Aerosil™ products from Evonik Industries, Germany), and starches.

### Coloring Agents:

Coloring agents can be used to color code the composition, for example, to indicate the type and dosage of the therapeutic agent therein. Suitable coloring agents include, without limitation, natural and/or artificial compounds such as FD&C coloring agents, natural juice concentrates, pigments such as titanium oxide, silicon dioxide, iron oxides, and zinc oxide, combinations thereof, and the like.

### Lubricants:

An effective amount of any generally accepted pharmaceutical tableting lubricant can be added to assist in compressing tablets. Useful tablet lubricants include magnesium stearate, glyceryl monostearates, palmitic acid, talc, carnauba wax, calcium stearate sodium, sodium or magnesium lauryl sulfate, calcium soaps, zinc stearate, polyoxyethylene monostearates, calcium silicate, silicon dioxide, hydrogenated vegetable oils and fats, stearic acid and combinations thereof.

### Glidants:

One or more glidant materials, which improve the flow of powder blends and minimize the dosage form weight variation, can be used. Useful glidants include but are not limited to silicone dioxide, talc and combinations thereof.

### Solvents:

Solvents that can be used in processing to form pharmaceutical compositions include but are not limited to water, methanol, ethanol, acidified ethanol, acetone, diacetone, polyols, polyethers, oils, esters, alkyl ketones, methylene chloride, isopropyl alcohol, butyl alcohol, methyl acetate, ethyl acetate, isopropyl acetate, castor oil, ethylene glycol monoethyl ether, diethylene glycol monobutyl ether, diethylene glycol monoethyl ether, dimethylsulphoxide, N,N-dimethylformamide, tetrahydrofuran and mixtures thereof.

The final formulations may be coated or uncoated. For coating, excipients such as film forming polymers, plasticizers, antiadherents and opacifiers can be used.

### Film-Forming Agents:

Various useful film-forming agents include but are not limited to cellulose derivatives such as soluble alkyl- or hydroalkylcellulose derivatives such as methylcelluloses, hydroxymethyl celluloses, hydroxyethyl celluloses, hydroxypropyl celluloses, hydroxymethyethyl celluloses, hydroxypropyl methylcelluloses, sodium carboxymethyl celluloses, etc., acidic cellulose derivatives such as cellulose acetate phthalates, cellulose acetate trimellitates and methylhydroxypropylcellulose phthalates, polyvinyl acetate phthalates, etc., insoluble cellulose derivative such as ethylcelluloses and the like, dextrins, starches and starch derivatives, polymers based on carbohydrates and derivatives thereof, natural gums such as gum Arabic, xanthans, alginates, polyacrylic acid, polyvinyl alcohols, polyvinyl acetates, polyvinylpyrrolidones, polymethacrylates and derivatives thereof (Eudragit™), chitosan and derivatives thereof, shellac and derivatives thereof, waxes and fat substances.

If desired, the films may contain additional adjuvants for coating processing such as plasticizers, polishing agents, colorants, pigments, antifoam agents, opacifiers, antisticking agents, and the like.

In addition to above coating ingredients, sometimes pre-formulated coating products such as Opadry™ Brown 03B86854 or Opadry II Blue (HPMC 2910/hypromellose 6 cP, titanium dioxide, lactose monohydrate, macrogol/polyethylene glycol 3000, triacetin, FD&C Blue/indigo carmine aluminum) supplied by Colorcon may conveniently be used. The products sold in dry form require only mixing with a liquid before use.

In embodiments of the present invention, equipment suitable for processing the pharmaceutical compositions include any one or more of mechanical sifters, granulator, blenders, roller compactors, compression machines, rotating bowls or coating pans, fluid bed processors, etc.

In embodiments of the present invention, the pharmaceutical compositions may be processed using any techniques such as direct compression, dry granulation, and wet granulation.

In embodiments, the present invention provides pharmaceutical compositions of eszopiclone or its salts in modified release dosage forms.

In embodiments the present invention is directed to processes for preparing pharmaceutical compositions comprising a therapeutically effective amount of eszopiclone or its pharmaceutically acceptable salts and at least one pharmaceutically acceptable excipient, a specific embodiment comprising:

a) sifting active ingredient;

b) sifting excipients;

c) dry mixing active and excipients in a granulator;

d) optionally compacting the step c) mixture and subsequently milling the compacts and sifting;

e) dissolving or dispersing a binder in a suitable solvent;

f) optionally granulating the step c) mixture using binder solution or dispersion from step e);

g) drying granules;

h) sifting granules from d) or g) and extragranular excipients;

i) blending sized granules or dry mixed excipients from step c) or milled and sifted granules from step d) with sifted extragranular excipients (except lubricants);

j) adding sifted lubricant to step i) and blending further; and

k) filling lubricated blend into hard gelatin capsules, or alternatively compressing into tablets and optionally filling tablets into capsules.

In another embodiment a process according to the invention for preparing a solid pharmaceutical composition of eszopiclone or a salt thereof comprises:

(i) dry mixing the drug or a salt thereof with at least one pharmaceutical excipient; and

(ii) dry processing the mixture obtained in step (i) into a desired solid form.

The dosage forms prepared by the above process can be tested for physical parameters such as weight variation, hardness, disintegration, friability etc. Several devices can be used to test tablet hardness such as a Monsanto tester, a Strong-Cobb tester, a Pfizer tester, a Erweka tester and a Schleuniger tester etc. Friability can be determined by a Roche friabilator, such as for 100 revolutions at 25 rpm. Disintegration testing for tablets can be performed in a USP tablet disintegration tester wherein a tablet is placed in basket, which moves upward and downward in a 1 L beaker of water at 37° C.

The dosage forms prepared by the above process can be subjected to in vitro dissolution evaluations according to Test 711 “Dissolution” in United States Pharmacopoeia 29, United States Pharmacopeial Convention, Inc., Rockville, Md., 2005 (“USP”) to determine the rate at which the active substance is released from the dosage forms, and the content of the active substance can conveniently be determined in solutions using techniques such as high performance liquid chromatography.

The present invention includes the use of packaging systems for multiple dose packaging such as high-density polyethylene containers, high-density polyethylene containers with polypropylene/low-density polyethylene closures, low-density polyethylene (LDPE) and or polypropylene and/or glass, and blisters or strips composed of aluminium of high-density polypropylene, polyvinyl chloride, polyvinylidene dichloride, aluminum/aluminum blisters with laminated desiccant system and polyamide packing. In another embodiment of the present invention, the packaging may also comprise various desiccants such as silica gel bags, molecular sieves and so on, which will enable the compositions to maintain their ERH levels.

In another embodiment the present invention provides pharmaceutical compositions comprising eszopiclone or its salts, wherein the percentage of active ingredient in the total compositions is in the range of from about 1% to about 10% w/w.

Certain specific aspects and embodiments of this invention are described in further detail by the examples below, which examples are provided solely for purposes of illustration and are not intended to limit the scope of the invention in any manner.

### EXAMPLE 1

### Solubility of Eszopiclone in Various Solvents

Procedure:

1) 100 mL of the solvent was placed into a 250 mL volumetric flask.

2) 10 mg of eszopiclone was added to the flask.

3) The flask contents were mixed for 24 hours using a rotary shaker to dissolve the eszopiclone completely.

4) Eszopiclone was added at room temperature to the clear solution of 3) in small amounts (up to about 100 mg) until undissolved eszopiclone was noted after shaking.

5) The flask was subjected to shaking for an additional 24 hours.

6) Presence of insoluble eszopiclone was checked and the pH of the solution was recorded.

7) A portion of step 6) solution was subjected to centrifugation until a clear solution was obtained, and/or was filtered through an ASTM #40 mesh sieve.

8) Content of the drug substance was determined using HPLC, comparing with a standard solution prepared preferably in the same solvent.

9) Amount of the drug substance dissolved was calculated as a percentage (weight/volume):

Q=(A×C×100×V2×V4×100)÷(B×V1×V3×Wt).

A=Response of the test solution.

B=Response of standard solution.

C=Concentration of standard solution in mg/mL.

Wt=Total amount of eszopiclone added, in mg.

V1=Volume of filtered test solution taken, in mL.

V2=Diluted volume, in mL.

V3=Volume of diluted test solution.

V4=Diluted volume, in mL.

The actual amount of drug substance dissolved in mg is:

m=(Q×Wt)÷100.

The solubility of the drug substance in mg/ml in the solvent medium tested is: S=m÷100.

Saturated solubility was determined in solvents as described below.

| Solvent | Solubility (mg/mL) |
|---|---|
| 0.1N Hydrochloric acid (pH 1.2) | 39.11 |
| 0.01N HCl | 4.18 |
| Acetate buffer (pH 4.5) | 11.62 |
| Phosphate buffer (pH 6.8) | 0.68 |
| Phosphate buffer (pH 7.5) | 0.33 |
| Simulated gastric fluid (pH 1.2) | 8.18 |
| 0.5% Sodium lauryl sulphate in water | 1.83 |
| Acid buffer (pH 1)* | 23.02 |
| Water | 0.3 |

*Acidic buffer is obtained by dissolving potassium chloride in 500 mL of water and adjusting the pH to 1 with concentrated hydrochloric acid.

### EXAMPLE 2

### Excipient Compatibility Studies of Eszopiclone and Stability

Study procedure:

1) Eszopiclone and excipient in the stated ratios were mixed well and sifted through an ASTM #40 mesh sieve.

2) The mixture was placed into 20 mL amber glass vials and stored under conditions of 40° C. and 75% relative humidity (RH), or at 50° C., for 1 month. Impurity analyses were performed before and after storage, and values in the table below are percentages of the initial eszopiclone content.

| | | | | 1 Month | | | |
|---|---|---|---|---|---|---|---|
| | | | | 40° C./ | | | |
| | Drug:Excipient | Initial | | 75% RH | | 50° C. | |
| Drug/Excipient Mixture | Wt. Ratio | Imp | T.I. | Imp | T.I. | Imp | T.I. |
| Eszopiclone/Dicalcium | 1:10 | 0.06 | 0.12 | 0.44 | 0.29 | 1.23 | 2.51 |
| phosphate | | | | | | | |
| Eszopiclone/Avicel ™ PH102 | 1:20 | 0.01 | 0.04 | 1.52 | 1.04 | 0.19 | 0.36 |
| Eszopiclone/HPMC 3 cps | 1:2 | 0.01 | 0.04 | 0.04 | 0.01 | 0.09 | 0.15 |
| Eszopiclone/Colloidal silicon | 1:1 | 0.02 | 0.05 | 0.52 | 0.35 | 1.22 | 2.02 |
| dioxide | | | | | | | |
| Eszopiclone/Croscarmellose | 1:1 | 0.01 | 0.05 | 0.08 | 0.03 | 0.047 | 0.08 |
| Eszopiclone/Magnesium | 1:0.5 | 0.01 | 0.04 | 0.07 | 0.03 | 0.021 | 0.04 |
| stearate | | | | | | | |
| Eszopiclone/Opadry ™ Blue | 1:1 | 0.01 | 0.06 | 0.13 | 0.07 | 0.09 | 0.15 |
| Eszopiclone/Pearlitol ™ SD | 1:10 | 0.01 | 0.02 | 0.3 | 0.2 | 0.3 | 0.44 |
| 200 | | | | | | | |
| Eszopiclone/Avicel PH101 | 1:20 | 0.02 | 0.05 | 0.19 | 0.11 | 0.38 | 0.55 |
| Eszopiclone/DCL21 | 1:10 | 0.01 | 0.29 | 0.16 | 0.09 | 0.18 | 0.29 |
| Eszopiclone | — | 0.01 | 0.02 | 0.47 | 0.33 | 0.35 | 0.5 |

Imp = 5-chloro-2-aminopyridine.
T.I. = Total impurities.

### EXAMPLE 3

### Pharmaceutical Compositions Comprising 1 mg and 3 mg of Eszopiclone

mg/Tablet

| | | |
|---|---|---|
| Ingredient | 3A | 3B |

Dry Mix

| | | |
|---|---|---|
| Eszopiclone | 1 | 3 |
| Microcrystalline cellulose | 75.85 | 73.85 |
| (Avicel RQ102) | | |
| Dibasic calcium phosphate | 16.15 | 16.15 |
| anhydrous (A-Tab ™)⁺ | | |
| Croscarmellose sodium | 5 | 5 |
| Colloidal silicon dioxide | 1 | 1 |
| (Aerosil ™ 200) | | |
| Magnesium stearate | 1 | 1 |

Coating

| | | |
|---|---|---|
| Opadry II Blue (33G20848)^{$} | 3 | — |
| Opadry II Blue (33G90615)^{#} | — | 3 |
| Water* | q.s. | q.s. |

*Evaporates during processing.
⁺A-Tab is a product of Rhodia.
^{$}Opadry II Blue (33G20848) is a formulated coating composition containing hypromellose, titanium dioxide, lactose monohydrate, macrogol, triacetin, and FD&C Blue No. 2.
^{#}Opadry II Blue (33G90615) is a formulated coating composition containing hypromellose, titanium dioxide, lactose monohydrate, macrogol, triacetin, and FD&C Blue No. 2.

Manufacturing procedure:

1. Avicel RQ 102 was sifted through an ASTM #40 mesh sieve.

2. Avicel RQ 102 and eszopiclone were mixed geometrically and sifted through an ASTM #40 mesh sieve.

3. Croscarmellose sodium and dibasic calcium phosphate were sifted through an ASTM #40 mesh sieve.

4. Ingredients of step 3 were added to step 2 and the blend was sifted through an ASTM #40 mesh sieve.

5. Aerosil 200 and magnesium stearate were sifted through ASTM #40 mesh and ASTM #60 mesh sieves, respectively.

6. The blend of step 5 was transferred to a double cone blender (1.5 L capacity) and blended for 15 minutes.

7. Aerosil 200 was added to the blender and blended for 2 minutes.

8. Magnesium stearate was added to the blender and blended for 5 minutes.

9. The lubricated blend was compressed into tablets.

10. Opadry II Blue 33G20848 (Opadry II Blue 33G90615 for 3 mg) was dispersed in water and stirring continued for about 45 minutes. The tablets were coated using this dispersion.

Drug release testing was conducted using the USP procedure and the following conditions: medium 0.1 N HCl; apparatus Type 2; stirring 50 rpm. Results are below.

Cumulative % of
Drug Dissolved

| Minutes | 3A | 3B |
|---|---|---|
| 10 | 86 | 84 |
| 15 | 87 | 85 |
| 20 | 90 | 88 |
| 30 | 93 | 91 |
| 45 | 96 | 94 |

The coated tablets were packaged in closed HDPE containers and stored for 3 months at 40° C. and 75% RH, and the analytical data for drug-related impurities are below, where impurity compound values are percentages of the label eszopiclone content, water content is expressed as percent of the composition, and dissolution is the percentage of contained eszopiclone that dissolved into 0.1 N HCl in 30 minutes.

| | 3A | | 3B | |
|---|---|---|---|---|
| Analysis | Initial | 3 Months | Initial | 3 Months |
| Impurity A | 0.0094 | 0.0232 | 0.0028 | 0.0190 |
| Impurity B | 0.0174 | 0.0423 | 0.0145 | 0.0264 |
| Impurity C | ND | ND | ND | ND |
| Impurity D | 0.0263 | 1.1509 | 0.0305 | 0.5944 |
| Highest | 0.0085 | 0.2183 | 0.0084 | 0.096 |
| Unidentified | | | | |
| Impurity | | | | |
| Total Impurities | 0.0728 | 1.4940 | 0.0755 | 0.7893 |
| Water by KF (%) | 4.8 | — | 4.7 | — |
| Dissolution (%) | 93 | — | 91 | — |

ND = Not detected.

### EXAMPLE 4

### Pharmaceutical Compositions Comprising 1 mg and 3 mg of Eszopiclone

(mg/Tablet)

| | | |
|---|---|---|
| Ingredient | 4A | 4B |

Dry Mix

| | | |
|---|---|---|
| Eszopiclone | 1 | 3 |
| Microcrystalline cellulose | 65.95 | 64 |
| (Avicel ™ RQ102) | | |
| Mannitol (Pearlitol ™ SD200) | 27 | 26.95 |
| Croscarmellose sodium | 3.3 | 3.3 |
| Colloidal silicon dioxide (Aerosil | 1.5 | 1.5 |
| 200) | | |
| Magnesium stearate | 1.25 | 1.25 |

Coating

| | | |
|---|---|---|
| Opadry ™ II Blue (33G20848) | 3 | — |
| Opadry II Blue (33G90615) | — | 3 |
| Water* | q.s. | q.s. |

*Evaporates during processing.

Manufacturing process:

1) Eszopiclone and Avicel RQ 102 were sifted through an ASTM #40 mesh sieve, mixed geometrically, and again sifted through an ASTM #40 mesh sieve.

2) Croscarmellose sodium and mannitol were sifted through an ASTM #40 mesh sieve.

3) Step 2) was added to step 1) and the blend was sifted through an ASTM #40 mesh sieve.

4) Aerosil 200 and magnesium stearate were sifted through ASTM #40 and #60 mesh sieves, respectively.

5) The dry mixture of step 3) was transferred into a double cone blender and blended for about 15 minutes.

6) Aerosil 200 was added to the blender and blended for 2 minutes.

7) Magnesium stearate was added to the blener and blended for 5 minutes.

8) The lubricated blend was compressed into tablets.

9) Opadry II Blue 33G20848 (or Opadry II Blue 33G90615 for 3 mg) was dispersed in water and stirring continued for about 45 minutes. The tablets were coated using this dispersion.

Drug release testing was conducted as described in Example 3, and the results are below.

Cumulative % of
Drug Dissolved

| Minutes | 4A | 4B |
|---|---|---|
| 10 | 90 | 80 |
| 15 | 92 | 82 |
| 20 | 93 | 85 |
| 30 | 93 | 88 |
| 45 | 93 | 92 |

The coated tablets were packaged in closed HDPE containers and stored for 3 months at 40° C. and 75% RH, and the analytical data (as in Example 3) are below.

| | 4A | | 4B | |
|---|---|---|---|---|
| Analysis | Initial | 3 Months | Initial | 3 Months |
| Impurity A | 0.0049 | 0.0282 | 0.003 | 0.0091 |
| Impurity B | 0.0144 | 0.0291 | 0.0188 | 0.0306 |
| Impurity C | ND* | ND | ND | ND |
| Impurity D | 0.0105 | 0.9570 | 0.0256 | 0.3492 |
| Highest | 0.0089 | 0.1549 | 0.0096 | 0.0605 |
| Unidentified | | | | |
| Impurity | | | | |
| Total Impurities | 0.0561 | 1.1995 | 0.0648 | 0.4818 |
| Water by KF (%) | 4.2 | — | 3.8 | — |
| Dissolution (%) | 93 | — | 88 | — |

*ND = Not detected.

### EXAMPLE 5

### Pharmaceutical Compositions Comprising Eszopiclone 1 mg and 3 mg

Grams

| | | |
|---|---|---|
| Ingredient | 5A | 5B |

Dry Mix

| | | |
|---|---|---|
| Eszopiclone | 20 | 60 |
| MicroceLac ™ 100^{@} | 1892 | 1852 |
| Croscarmellose sodium | 66 | 66 |
| Magnesium stearate | 22 | 22 |

Coating

| | | |
|---|---|---|
| Opadry II Blue (33G20848) | 60 | — |
| Opadry II Blue (33G90615) | — | 60 |
| Water* | 88 | 88 |

*Evaporates during processing.
^{@}MicroceLac 100 supplied by Molkerei MEGGLE Wasserberg GmbH & Co. KG, Wasserburg, Germany.

Manufacturing process:

1) MicroceLac 100 was divided into three parts: part 1 (200 g), part 2 (400 g) and the remaining quantity as part 3.

2) Eszopiclone and part 1 of MicroceLac 100 were mixed together and sifted through an ASTM #40 mesh sieve.

3) Step 2) materials were geometrically mixed with part 2 of Microcelac 100.

4) Part 3 of MicroceLac 100 was mixed geometrically with step 3) materials and the mixture was sifted through an ASTM #40 mesh sieve.

5) Croscarmellose sodium was sifted through an ASTM #40 mesh sieve.

6) Step 4) and 5) materials were combined and together sifted through an ASTM #40 mesh sieve.

7) Magnesium stearate was sifted through an ASTM #60 mesh sieve

8) The mixture of step 6) was transferred into a double cone blender and blended for about 15 minutes.

9) Magnesium stearate was added to the blender and blended for about 5 minutes.

10) The lubricated blend from step 9) was compressed into tablets of 100 mg average weight.

11) Opadry II Blue 33G20848 (or Opadry II Blue 33G90615 for 3 mg) was dispersed in water and stirring continued for about 45 minutes. The tablets were coated using this dispersion.

Drug release testing was conducted as in Example 3, and the results are below.

Cumulative % of
Drug Dissolved

| Minutes | 5A | 5B |
|---|---|---|
| 10 | 96 | 95 |
| 15 | 96 | 95 |
| 20 | 96 | 95 |
| 30 | 96 | 95 |
| 45 | 96 | 95 |

The coated tablets were packaged in closed HDPE containers and stored for 3 months at 40° C. and 75% RH, and the analytical data (as in Example 3) are below.

| | | | | | LUNESTA | | LUNESTA | |
|---|---|---|---|---|---|---|---|---|
| | 5A | | 5B | | 1 mg | | 3 mg | |
| Analysis | Initial | 3 Months | Initial | 3 Months | Initial | 3 Months | Initial | 3 Months |
| Impurity A | 0.0072 | 0.0263 | 0.0028 | 0.0186 | 0.0757 | 0.1182 | 0.0207 | 0.0211 |
| Impurity B | 0.0046 | 0.0204 | 0.0034 | 0.0125 | 0.0122 | 0.0653 | 0.0092 | 0.0621 |
| Impurity C | 0.0029 | ND | ND | ND | ND | ND | ND | ND |
| Impurity D | 0.0033 | 0.252 | 0.0021 | 0.1182 | 0.0231 | 0.4057 | 0.0261 | 0.4672 |
| Highest | 0.0152 | 0.1073 | 0.0084 | 0.0251 | 0.0118 | 0.0624 | 0.0134 | 0.0820 |
| Unidentified | | | | | | | | |
| Impurity | | | | | | | | |
| Total | 0.0515 | 0.5311 | 0.0264 | 0.2217 | 0.1334 | 0.7244 | 0.0874 | 0.7106 |
| Impurities | | | | | | | | |
| Water by KF | 6.13 | — | 5.89 | — | 4.3765 | 7.67 | 4.4337 | 4.92 |
| (%) | | | | | | | | |
| Dissolution | 96 | — | 95 | — | 95 | 92 | 93 | 97 |
| (%) | | | | | | | | |

### EXAMPLE 6

### Pharmaceutical Compositions Comprising Eszopiclone 1 mg, 2 mg, and 3 mg

| | mg/Tablet | | |
|---|---|---|---|
| Ingredient | 6A | 6B | 6C |
| Eszopiclone | 1 | 2 | 3 |
| MicroceLac 100 | 94.6 | 93.9 | 92.9 |
| Croscarmellose sodium | 3.3 | 3.3 | 3.3 |
| Magnesium stearate | 0.8 | 0.8 | 0.8 |
| Opadry II Blue | 3 | 3 | 3 |
| Water* | 18.4 | 18.4 | 18.4 |

*Evaporates during processing.

Manufacturing process:

1) MicroceLac 100 was divided into two parts.

2) Eszopiclone and MicroceLac 100 part 1 were mixed together and co-sifted through an ASTM #40 mesh sieve.

3) MicroceLac 100 part 2 was added to step 2), co-sifted through an ASTM #40 mesh sieve, and mixed in a polyethylene bag.

4) Croscaramellose sodium was sifted through an ASTM #40 mesh sieve and added to step 3).

5) The step 4) mixture was placed into a double cone blender and blended uniformly for 15 minutes at a speed of 20 rpm.

6) Magnesium stearate was sifted through an ASTM #60 mesh sieve, added to step 5), and blended uniformly for 5 minutes in a double cone blender.

8) Step 6) was compressed into tablets with 6.55 mm round, biconcave punches.

9) Opadry II Blue was dispersed in water and mixed continuously to form a suspension.

10) Step 8) tablets were coated with step 9) coating to produce a weight gain of 2.5% to 3%.

Physical parameters of prepared tablets are given below.

| Parameter | 6A | 6B | 6C |
|---|---|---|---|
| Hardness (Kiloponds) | 3-8 | 3-8 | 3-8 |
| Disintegration time (minutes) (in water) | ≦4 | ≦4 | ≦4 |
| Eszopiclone content uniformity (% relative | 1.8 | 1.8 | 1.8 |
| standard deviation) | | | |

Products containing 1 mg and 3 mg of eszopiclone were packaged in closed HDPE bottles, were stored at 40° C. and 75% RH for 2 months, and were analyzed at intervals to determine their impurity content. A commercial product was also similarly tested for comparison. Samples also were stored unpackaged at 40° C. and 75% RH for 2 weeks. Impurity contents in the following table are expressed as weight percentages of the label eszopiclone content.

| | LUNESTA | | | | LUNESTA | | | |
|---|---|---|---|---|---|---|---|---|
| | 1 mg | | 6A | | 3 mg | | 6C | |
| Time | Imp | T.I. | Imp | T.I. | Imp | T.I. | Imp | T.I. |
| Initial | 0.023 | 0.13 | 0.03 | 0.1 | 0.026 | 0.08 | 0.023 | 0.04 |
| 2 weeks (unpackaged) | — | — | 0.08 | 0.15 | — | — | 0.035 | 0.08 |
| 1 Month | 0.02 | 0.23 | 0.13 | 0.22 | 0.013 | 0.17 | 0.05 | 0.11 |
| 2 Months | — | — | 0.37 | 0.5 | — | — | 0.16 | 0.26 |

Imp = 5-chloro-2-aminopyridine.
T.I. = Total impurities.

We claim:

1. A pharmaceutical composition comprising: a) eszopiclone or a salt thereof; and b) microcrystalline cellulose, lactose monohydrate, or a combination thereof.

2. The pharmaceutical composition of claim 1, comprising both of microcrystalline cellulose and lactose monohydrate

3. The pharmaceutical composition of claim 1, wherein a weight ratio of eszopiclone to a total of microcrystalline cellulose and lactose monohydrate ranges from about 1:5 to about 1:100.

4. The pharmaceutical composition of claim 1, prepared using eszopiclone having an average particle size less than about 50 μm.

5. The pharmaceutical composition of claim 1, prepared using eszopiclone having a particle size distribution where D₉₀ is not more than about 10 μm, D₅₀ is not more than about 8 μm, and D₁₀ is not more than about 4 μm.

6. The pharmaceutical composition of claim 1, having a moisture content less than about 8 percent by weight.

7. The pharmaceutical composition of claim 1, wherein the 5-chloro-2-aminopyridine impurity content is less than about 2 percent of the label eszopiclone content.

8. The pharmaceutical composition of claim 1, wherein the 5-chloro-2-aminopyridine impurity content is less than about 1 percent of the label eszopiclone content.

9. The pharmaceutical composition of claim 1, wherein the 5-chloro-2-aminopyridine impurity content is less than about 0.5 percent of the label eszopiclone content.

10. The pharmaceutical composition of claim 1, wherein the content of any single drug-related impurity is less than about 0.5 percent of the label eszopiclone content.

11. The pharmaceutical composition of claim 1 comprising eszopiclone, wherein the total drug-related impurity content is less than about 0.5 percent by weight of the label eszopiclone content.

12. A pharmaceutical tablet composition comprising eszopiclone or a salt thereof and at least one pharmaceutically acceptable excipient, which composition does not contain calcium phosphate.

13. The pharmaceutical tablet composition of claim 12, comprising: a) eszopiclone; b) a spray-dried composition of about 75 percent lactose monohydrate and about 25 percent microcrystalline cellulose, by weight; c) a disintegrant; d) a lubricant; and, optionally, e) a binder.

14. The pharmaceutical tablet composition of claim 12, wherein eszopiclone is present in an amount less than about 15 percent by weight of the composition.

15. The pharmaceutical tablet composition of claim 12, being prepared using wet granulation, dry granulation, or direct compression.

16. A pharmaceutical tablet composition comprising eszopiclone and a spray-dried composition of about 75 percent lactose monohydrate and about 25 percent microcrystalline cellulose, by weight.

17. The pharmaceutical tablet composition of claim 16, further comprising a disintegrant and a lubricant.

18. The pharmaceutical tablet composition of claim 16, further comprising croscarmellose sodium and a lubricant.

19. The pharmaceutical composition of claim 16, wherein the total drug-related impurity content is less than about 0.5 percent by weight of the label eszopiclone content.

20. The pharmaceutical composition of claim 16, having a moisture content less than about 8 percent by weight.
